SEQUENCE LISTING (6 sequences)

SEQ ID NO: 1 - DNA, 19 bp, Artificial Sequence
  SP-106F3
ccgtttgttaaatcaggct

SEQ ID NO: 2 - DNA, 22 bp, Artificial Sequence
  SP-106B3
ggtattgattggtcacaatgtc

SEQ ID NO: 3 - DNA, 48 bp, Artificial Sequence
  SP-106FIP
gcgagtgcggttataaattctctatttttctacacagacactcgacaa

SEQ ID NO: 4 - DNA, 49 bp, Artificial Sequence
  SP-106BIP
ttcttacctcaaatttccgcaacttttggaaaccagtaatagtagtcaa

SEQ ID NO: 5 - DNA, 22 bp, Artificial Sequence
  SP-106LF
aggacaaggtttgattgaggac

SEQ ID NO: 6 - DNA, 22 bp, Artificial Sequence
  SP-106LB
catcaaggatttctgttaccaa

(19) United States
(12) Patent Application Publication
(10) Pub. No.: US 2025/0290157 A1
(43) Pub. Date: Sep. 18, 2025

(54) PATHOGEN TESTING DEVICE

(71) Applicant: RT MICROFLUIDICS, INC, Brookline, MA (US)

(72) Inventors: David Wolf, Maynard, MA (US); Dylan A. Bulseco, Princeton, MA (US); Olivia Mamane, Brookline, MA (US); Nathalya Mamane; Lucas Hamilton-Boureza, Brookline, MA (US); Labiba Tashin, Brookline, MA (US); James Kusardi, Brookline, MA (US)

(21) Appl. No.: 18/576,065

(22) PCT Filed: Jun. 30, 2022
(86) PCT No.: PCT/US2022/073307

§ 371 Date: Jan. 2, 2024

Related U.S. Application Data
(60) Provisional application No. 63/218,023, filed on Jul. 2, 2021.
(60) Provisional application No. 63/243,839, filed on Sep. 14, 2021.

(51) Int. Cl. C12Q 1/689; C12Q 1/14

(52) U.S. Cl. CPC C12Q 1/689; C12Q 1/14

(57) ABSTRACT

A method of detecting a pathogen in a fluid sample includes adding the fluid sample to a first region of an assembly; amplifying a polynucleotide sequence of a pathogen in the first region using an amplification method; and detecting a presence of the pathogen by one of the following. Identifying a release of a dye from a pH sensitive polymer. Or, identifying a release of a dye from the first region to a second region that are separated by a pH sensitive polymer. Or, activating a pH sensitive dye in the first region to indicate a presence of the pathogen. Or, indicating a presence of the pathogen on a lateral flow assay in a second region that is separated from the first region by a pH sensitive polymer. Each identification, activation, or indication step occurs after the amplification method adjusts a pH of the fluid sample.

### CROSS-REFERENCE

This application is the U.S. National Stage entry of International Application No. PCT/US2022/073307, filed Jun. 30, 2022, which claims the benefit of and priority to U.S. Provisional Application No. 63/243,839, filed Sep. 14, 2021, and U.S. Provisional Application No. 63/218,023, filed Jul. 2, 2021, the disclosures of each are hereby incorporated by reference in their entireties for all purposes.

### SEQUENCE LISTING

The application contains a Sequence Listing in computer readable form. The computer readable form is incorporated herein by reference. Said ASCII copy, created on Aug. 7, 2024, is named RTMC-001WOUS_SL.txt and is 1,426 bytes in size.

### TECHNICAL FIELD

This disclosure relates to a pathogen testing assembly that determines the presence of a pathogen on a chip. Also disclosed is a method of determining a pathogen using a chip-based pathogen testing assembly.

### BACKGROUND

A wide range of testing devices have been derived to determine whether a patient is infected with a pathogen. For example, blood, saliva, urine, and/or stool samples can be taken from a patient and tested to determine whether the DNA and/or RNA of a pathogen is present by using various known markers bound to a sample of a user. These techniques use a wide range of testing devices that often include sending the sample to a laboratory for analysis by a professional or insertion of a sample into a specifically configured analytical instrument to get an output of whether a patient's blood, saliva, urine, and/or stool contains a pathogen. In the case of Streptococcus pyogenes, a widely used test is a throat culture. In short, a swab of the throat in a patient is taken, allowed to grow in a specific receptacle, and the presence of the specific bacterium is detected using a microscope, chemical test, or both. Other techniques include rapid antigen tests, but these tests lack sufficient specificity and/or accuracy to give the patient a reliable answer on whether a Streptococcus pyogenes infection is present.

What is needed is a test that can give rapid results to a person with sufficient accuracy and/or specificity to give the patient confidence on whether Streptococcus pyogenes is specifically present without the need for a doctor and/or additional facility for testing. What is needed is a test that can be performed by a person with little or no supervision by medical professionals.

### SUMMARY

The present disclosure provides assemblies and methods for testing for presence of a pathogen from a fluid sample of a patient.

In one example, a method of detecting a pathogen in a fluid sample includes adding the fluid sample to a first region of an assembly; amplifying a polynucleotide sequence of a pathogen in the first region using an amplification method; and detecting a presence of the pathogen by one of the following steps: identifying a release of a dye from a pH sensitive polymer after the amplification method adjusts a pH of the fluid sample; or identifying a release of a dye from the first region to a second region after the amplification method adjusts a pH of the fluid sample, wherein the first and second regions are separated by a pH sensitive polymer; or activating a pH sensitive dye in the first region to indicate a presence of the pathogen as the amplification method progresses; or indicating a presence of the pathogen on a lateral flow assay in a second region that is separated from the first region by a pH sensitive polymer so that the fluid sample traverses the pH sensitive polymer after the amplification method adjusts a pH of the fluid sample.

In the above aspect, amplifying the polynucleotide sequence of the pathogen in the first region using the amplification method may include contacting the fluid sample with one or more amplification components and/or buffers and/or applying heat to the fluid sample to amplify the polynucleotide sequence. Amplifying the polynucleotide sequence of the pathogen in the first region using the amplification method may include contacting the polynucleotide sequence with a deoxyribonucleotide triphosphate compound so that acidity of the fluid sample is increased due to the amplification method. Amplifying the polynucleotide sequence of the pathogen in the first region using the amplification method may include contacting the polynucleotide sequence, one or more enzymes, one or more primers, and a deoxyribonucleotide triphosphate compound so that acidity of the fluid sample is increased due to the amplification method. The amplification method may include polymerase chain reaction or isothermal amplification. The isothermal amplification may include at least one of loop-mediated isothermal amplification; strand-displacement amplification; single primer isothermal amplification; strand exchange amplification; cross-priming amplification; helicase-dependent amplification; rolling circle amplification; multiple displacement amplification; recombinase polymerase amplification; or nucleic acid sequence-based amplification. The amplification method may include loop-mediated isothermal amplification. The pH sensitive polymer may include natural or semi-natural materials, synthetic acidic polymers, synthetic basic polymers, or any combination thereof. The dye may include one or more of an organic dye and/or a synthetic dye, a nanoparticle, conjugated nanoparticle, and/or a quantum dot. The dye may be attached to a carrier, and the carrier may include a protein, a polynucleic acid, a macromolecule, a virus particle, and/or a nanoparticle. The pathogen may be Streptococcus pyogenes.

In another aspect, an assembly for detecting a pathogen in a fluid sample includes a first region that performs an amplification method of a nucleotide of the pathogen, and the amplification method adjusts a pH of the fluid sample. The assembly includes an indicator for determining presence of the pathogen. The indicator includes at least one of the following. A dye that is positioned within a pH sensitive polymer or is pH sensitive and within the first region, and the dye indicates a presence of the pathogen as the amplification method adjusts the pH of the fluid sample. Or, a dye in a first region that is separated from the second region by a pH sensitive polymer so that the fluid sample is traversable across the pH sensitive polymer after the amplification method adjusts a pH of the fluid sample. Or, a lateral flow assay positioned within a second region, and the first and second regions are separated by a pH sensitive polymer so that the fluid sample is traverse-able across the pH sensitive polymer after the amplification method adjusts the pH of the fluid sample.

The assembly may further include a cover that has at least one portion that is transparent and positioned over the first and/or second region so that indication of the pathogen is visible by viewing through the cover. The assembly may include a heating element connected with the first region and configured to maintain a temperature or range of temperatures. The assembly may include one or more buffers that do not inhibit adjustment of pH of the fluid sample, primers that interact with the nucleotide sequence of the pathogen, lysing agents that treat the fluid sample, or any combination thereof.

In another aspect, the assembly includes a chip defining at least one microfluidic channel. The chip includes a first chamber that conducts an isothermal amplification process and a second chamber connected to the first chamber by a closable pathway. The chip includes at least one lateral flow assay positioned at an end of the second chamber free of contact with the closable pathway. The assembly includes a lysing agent configured to form a treated sample from a fluid sample of a patient and at least one assay material configured to be associated with the microfluidic channel and tags genetic material derived from the pathogen in the treated sample. The first chamber receives the treated sample, and the microfluidic chip indicates the presence of the pathogen from the combination of the treated sample and the at least one assay material at the lateral flow assay.

The lysing agent may be water and/or be a detergent including one or more of guanidine thiocyanate, lysozyme, lysostaphin, or any combination thereof. Before the lysing agent and the fluid sample of the patient are contacted, the lysing agent may be housed in an Eppendorf tube, a conical vial. The lateral flow assay may further include a conjugate pad having the assay material that includes gold nanoparticles and avidin, streptavidin, or neutravidin; a test line that indicates a presence of the pathogen by a first visual line; and a control line that indicates a presence of the gold nanoparticle and the avidin by a second visual line, the control line positioned downstream of the test line. The first chamber may have a shape of a serpentine channel. The first chamber and/or the second chamber may each independently have a shape of a circle, square, rectangle, triangular, oval, or any other shape sufficient to house fluids. The assembly may further include a separator positioned between the first and second chambers, and the separator may be releasably separated from the first and second chambers so that removing the separator from the assembly allows fluid communication between the first and second chambers. The first chamber may be in fluid communication with an aperture for receiving the treated sample at a first end and closable in fluid communication with the second chamber at the second end. The assembly may further include an amplification chamber adjacent to the first chamber, and the amplification chamber may include a buffer and/or an amplification component sufficient to assist with the isothermal amplification reaction. The amplification chamber may include one or more a blister packs containing chemical components that assist with the amplification reaction and that are activated by puncturing and/or rupturing the blister pack. The reagent chamber may be adjacent to and in closable fluid communication with the first and/or second chambers, and the reagent chamber may be set up to mix buffer with the treated sample after the treated sample exits the first chamber and enters the second chamber. The assembly may further include a reagent chamber adjacent to and in closable fluid communication with the second chamber, and the reagent chamber may be set up to mix buffer with the treated sample after the treated sample exits the first chamber and enters the second chamber. The reagent chamber may include a blister pack housing the buffer and activated by puncturing or rupturing the blister pack. The lysing agent and the treated sample may enter the chip at an inlet positioned adjacent to a location of the first chamber and the microfluidic channel, and wherein the treated sample flows across the lateral flow assay at an outlet positioned at a location adjacent to the second chamber.

In another aspect, the disclosure provides an assembly for testing for presence of a pathogen from a fluid sample of a patient including a lysing agent separate from the chip that forms a treated sample from a fluid sample of a patient. The assembly includes a chip that defines at least one microfluidic channel. The chip includes a first chamber that receives the treated sample and conduct an isothermal amplification process. The first chamber includes one or more component chambers that include one or more amplification components. The chip includes a second chamber connected to the first chamber by a first closable pathway and a reagent chamber including a buffer connected to the second chamber by a second closable pathway. The buffer improves flow of fluids through the second chamber. The chip includes an outlet including at least one assay material, and the at least one assay material tags genetic material derived from the pathogen in the treated sample. The outlet indicates the presence of the pathogen by the tag of the genetic material by a visual line.

The assembly may further comprise a reagent opening device pivotably connected with the chip, and the reagent opening device may include pointed surfaces that open the one or more component chambers to release an amplification component and to open the reagent chamber to release the at least one assay material. The at least one assay material may include one or more of buffers, gold nanoparticles, avidin, or a combination thereof, and the amplification component may include one or more of rehydration and reaction buffer mixes, magnesium acetate, primers, one or more reverse transcriptase enzymes, one or more DNA binding protein, one or more DNA polymerases, magnesium acetate or any combination thereof, and wherein the pathogen includes Streptococcus pyogenes.

In another aspect, the disclosure provides a method for detecting a pathogen in a patient. The method includes mixing a lysing agent and a fluid sample from a patient to form a treated sample and inserting the treated sample into a chip having a first and second chambers separated by a closable pathway. The treated sample is inserted into the first chamber. The method includes contacting an amplification component with the treated sample in the amplification chamber or the first chamber to form a mixture of the amplification component and the treated sample. The method includes opening the closable pathway to allow fluid flow between the first and second chambers. The method includes contacting a buffer with the mixture of the amplification component and the treated sample in the second chamber to form a mixture of the buffer, the amplification component, and the treated sample. The method includes contacting the mixture of the buffer, the amplification component, and the treated sample with a lateral material at a lateral flow assay to determine whether a pathogen is present by a control line and a test line.

In another aspect, the disclosure provides a method for detecting a pathogen in a patient. The method includes adding a treated sample into a chip having first and second chambers separated by a closable pathway. Then the treated sample is inserted into the first chamber. The method includes contacting an amplification component with the treated sample in the amplification chamber or the first chamber to form a mixture of the amplification component and the treated sample. The method includes opening the closable pathway to allow fluid flow between the first and second chambers. The method includes contacting a buffer with the mixture of the amplification component and the treated sample in the second chamber to form a mixture of the buffer, the amplification component, and the treated sample. The method includes contacting the mixture of the buffer, the amplification component, and the treated sample with a lateral material at a lateral flow assay to determine whether a pathogen is present by a control line and a test line.

The methods may include opening, after the inserting of the treated sample into the chip having the first and second chambers separated by the closable pathway, component and reagent chambers incorporated into the chip to release the amplification reagent from the component chamber and the assay material from the reagent chamber. The methods may be performed within partially or wholly within a single microfluidic channel of the chip.

The above disclosed assemblies and methods are described separately, and one or more of the above features may be used with another aspect depending on the desired configuration of the user.

The assemblies and methods described herein provide a test that gives a rapid and accurate result after receiving a bodily fluid sample from a user. The assemblies and methods are configured such that the user can quickly get a result from the bodily fluid so an immediate decision can be made on treatment for the user without the need to send the bodily fluid to through an additional machine or another facility for testing. For example, a user can test the bodily fluid and find out immediately whether antibiotics are appropriate without waiting a day or several hours to receive results. The assemblies and methods provide a technique for testing for a pathogen that can be administered in a setting with little or no medical supervision, such as virtually, at home, or in a pharmacy because all that is needed for an immediate determination can be packaged in an off the shelf consumer package.

### BRIEF DESCRIPTION OF THE DRAWING

FIG. 1 is a top view of an example of an assembly as disclosed herein.

FIG. 2 is a top view of an example of an assembly as disclosed herein.

FIG. 3 is a side view of an example of housing.

FIG. 4A is an exploded view of an assembly as disclosed herein.

FIG. 4B is an example of a top view of the chip of FIG. 4A.

FIG. 5 shows a set of functional primers to carry out the LAMP reaction for the detection of Streptococcus pyogenes

FIG. 6 shows the successful determination of Streptococcus pyogenes in human saliva using LAMP.

### DETAILED DESCRIPTION

The present assemblies and methods described herein include a fluidic system that can test for the presence of a pathogen without the need for long wait times or medical technician administration. The assemblies and methods provide a technique that can be utilized at home, virtually, within the patient room, at the pharmacy, or at a testing center designed to provide quick feedback to the patient so that a decision can be made whether a positive pathogen result is determined. The techniques and assemblies provide a technique that can be conducted by taking a fluid sample without or with little supervision from a medical profession and, thus, could be utilized to facilitate a preliminary healthcare decision based on the test result, as desired.

The present disclosure provides methods and assemblies for testing for presence of a pathogen from a fluid sample of a patient. The fluid sample may be a bodily fluid, such as saliva, blood, lymph, urine, spinal fluid, gastric fluid, pharyngeal, or mucosal secretion. The method and assembly include polymers that dissolve or become porous in response to protons (for example, H+ or H₃O+ ions) produced by the nucleotide amplification reaction. The assembly includes a first region where a fluid sample is added and amplification is conducted in the first region. In other examples, an alternative pre-amplification region is used to first mix the fluid sample with a lysing agent so that the fluid sample is treated before the amplification reaction.

In some examples, a polymer separates a first and second region and prevents the sample from flowing to the second region until the amplification method alters the pH of the fluid sample. As protons are produced via a reaction between the fluid sample of the patient and reactants associated with the amplification method and present in the first region and the pH drops in the reaction chamber, the polymer dissolves or becomes porous and allows liquids (i.e., the treated sample and/or reactants) to traverse the polymer and into the second region. The second region may include components sufficient to identify the presence of the pathogen, such as a lateral flow assay, or a dye that may traverse the polymer and mix with the fluid sample, which would indicate the presence of the pathogen due to a pH change that releases the polymer to the fluid sample. These components can be visual cues to the user that a pathogen is present in their sample.

In some examples, temperature may be used in conjunction with or separate from a pH to effect dissolution or increased permeability of the polymer. Raising temperature can be expected to increase the effect of pH on the polymer, and subsequently, the polymer will more quickly or more efficiently be dissolved, when compared to the process of dissolving at room temperature. Accordingly, the polymer pKa can be chosen such that the polymer has an increased dissolution at the temperature of the amplification reaction. Alternatively, once a period of time has elapsed for amplification to occur, the temperature can be further elevated to further promote dissolution of the polymer.

In the assemblies and techniques used in this application, the user has an indication either on a lateral flow assay or a color change or repositioning in dyes to indicate a positive result-meaning that the pathogen is present in the fluid sample. Where no positive indication is shown, the assemblies and techniques have not detected the presence of the pathogen. A nonlimiting example of a user as described in this disclosure may be as a patient, a parent, a medical provider, a lab technician, or any other user that can operate the assembly and/or method.

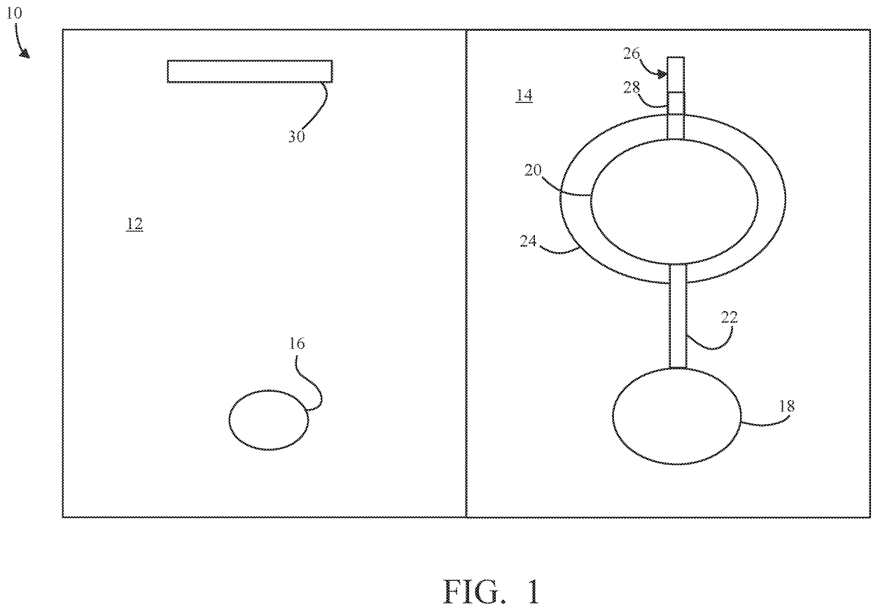

FIG. 1 is a top view of an example of an assembly 10 that is open. The assembly 10 is configured such that a fluid sample from a patient can be received by the assembly 10 for an implication method and/or subsequent identification of the presence of a pathogen in the fluid sample. The assembly 10 includes a first and second housings 12, 14 that are configured to rotatably connect relative to each other. In some examples, the first and second housings 12, 14 may fold like a card. In other examples, the first and second housings 12, 14 may be completely separate from each other such that before or after the addition of the fluid sample the first and second housings 12, 14 are sandwiched together. The first and second regions 12, 14 may be secured together by any means sufficient to prevent loss or contamination of the components within the assembly. For example, the first and second regions 12, 14 may be secured together using a tape, sealant, clip, hook and loop fastener, adhesive, glue, snap fit, friction fit, or any combination thereof. The first and second housings 12, 14 may be composed of any material sufficient to facilitate the amplification methods described herein.

For example, the first and second housing 12, 14 may be composed of paper, cardboard, metal, plastic, or any combination thereof. The first and second housings 12, 14 may include any additive sufficient to improve the properties of the first and second housings 12, 14 before, during, or after the amplification reaction progresses. For example, additives may be included to improve stiffness, acid resistance, UV resistance, flexibility, fluid flow, smoothness, roughness, color contrast, or any combination thereof.

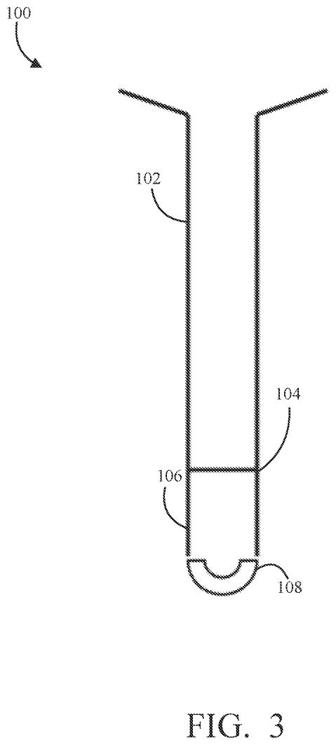

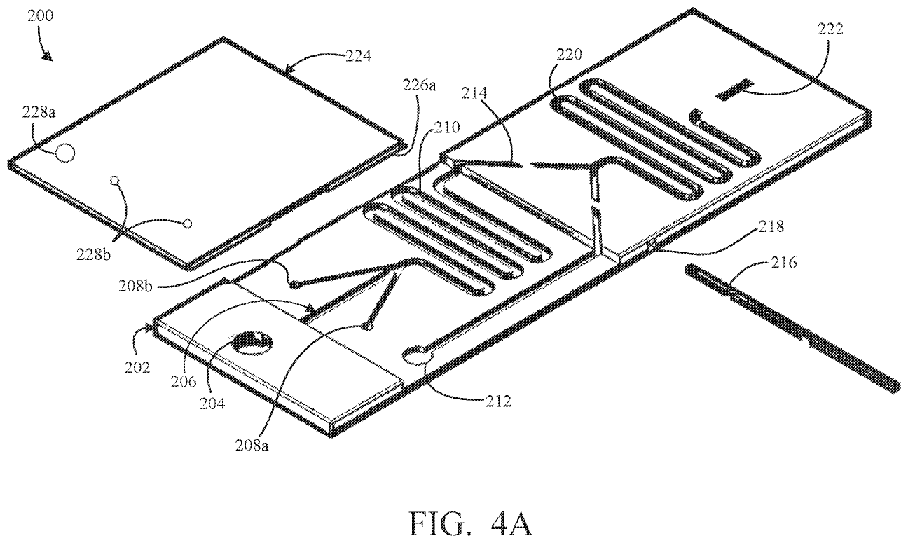

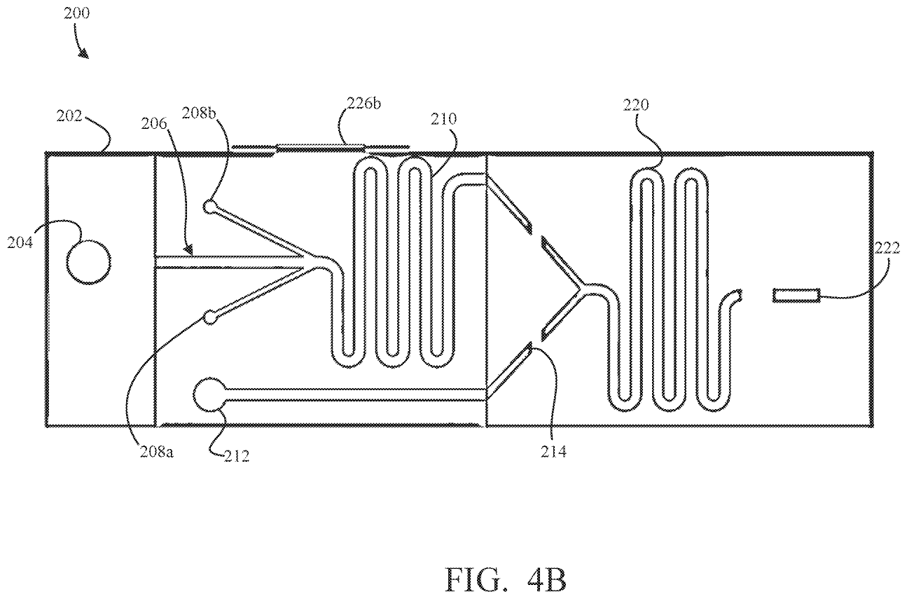

The assembly 10 further includes an input 16 that is positioned within the first housing 12 and is configured to receive a fluid sample. The input 16 may be positioned such that the fluid sample is directed to a pre-amplification region 18 that is positioned within the second housing 14. The input 16 may be similar to the input described in relation to FIGS. 4A-4B. The pre-amplification region 18 is configured to treat the fluid sample (e.g., using a detergent and/or lysing agent) before the fluid sample is subjected to the amplification methods described herein. In other examples, the fluid sample may be treated in separate container (not shown; see, for example FIG. 3), and then the fluid sample is added to the input 16, which directs the fluid sample directly to a first region 20 that is configured to facilitate the amplification method and/or reaction to determine presence of the pathogen.

The first region 20 is connected with the pre-amplification region 18 via a passage 22. The passage 22 may have an openable plug (not shown) that prevents mixture of components in the pre-amplification chamber 18 and the first region 20 until such time that the fluid sample is prepared and ready for the amplification method. At that point, the passage 22 may be opened and drain the fluid sample into the first region 20. The first region 20 is surrounded by a heating element 24 that is configured to attain or maintain a temperature sufficient to amplify the nucleotide sequence of a pathogen during the amplification method.

Positioned on an opposite side relative to the passage 22, a channel 26 extends from the first region 20 and fluid communication is prevented across the channel 26 by a plug 28. The plug 28 may have any configuration sufficient to separate the first region 20 from another region (e.g., a portion of the channel 26). For example, the plug 28 may have a configuration as a releasable plug that can be pulled by the user; a polymer that is pH sensitive relative to the amplification method; and/or any other removable or traversable component. A window 30 is positioned such that a user can see the channel 26 and determine if the pathogen sample has been positively identified in the fluid sample. For example, the pathogen may be identified by a color change to the fluid sample that is seen in the channel 26. In other examples, the plug 28 is configured as a polymer that is pH sensitive, and as the fluid sample reaches a pH sufficient to allow the plug 28 to dissolve and/or become porous during the amplification method, the fluid sample that has a color indicator traverses the polymer so that the user can see the fluid sample through window 30 in the portion of the channel 26 that is separated from the first region 20 by the plug 28.

The first region 20 may have any configuration sufficient to amplify the nucleotide sequence of a pathogen. For example, the first region 20 may include one or more openings that lead to one or more other regions used for identifying a pathogen, such as an opening at the channel 26. Another example of an opening would be any pathway leading to or from the input 16. Although shown in two dimensions, the first region 20 and/or the heating element 24 may have any three and/or two-dimensional shape sufficient to facilitate the progression of the amplification method. For example, the shape of the first region 20 may be determined based on the volume of the components and desired fluid sample for carrying out the amplification method.

In other examples, the first region 20 may include an insulator (not shown) positioned around a periphery of the first region 20 which assists with controlling the temperature changes during the amplification method. The insulator (not shown) may be configured such that it is positioned around the heating element 24 instead of the first region 20, and the insulator (not shown) may control temperature effects from the external environment to first region 20, the heating element 24, or both. The insulator (not shown) may include any material that is sufficient to control undesirable temperature fluctuations. The insulator (not shown) may include any compound that has a sufficiently low thermal conductivity to regulate the temperature of the first region 20. For example, the insulator (not shown) may include paper, a polymer, or a combination of both.

The first region 20 may include any component sufficient to promote an amplification reaction. The first region 20 may include any component sufficient to adjust the pH of the fluid sample and lead to an indication of the pathogen. The first region 20 may include any component that can assist with identification of a nucleotide sequence of a pathogen in a fluid sample. For example, the first region 20 may include one or more of primers, buffers, dyes, chemical heaters, betaine, detergent, additional divalent cations, dNTPs, conjugated dNTPs (e.g. biotin-labeled dNTP), various enzymes such as reverse transcriptase, strand displacing enzymes common in isothermal amplification, lysing agents, or any combination thereof. The first region 20 may include components in a solid state, liquid state, or both. For example, the first region 20 may have one portion configured as a blister pack that can be punctured or ruptured such that liquid elements in another portion of the first region 20 can mix and begin the amplification reaction. In some examples, some of the components are solid and some are liquid, and both the solid and liquid components are configured to mix with the fluid sample as the fluid sample enters the first region 20 from the input 16.

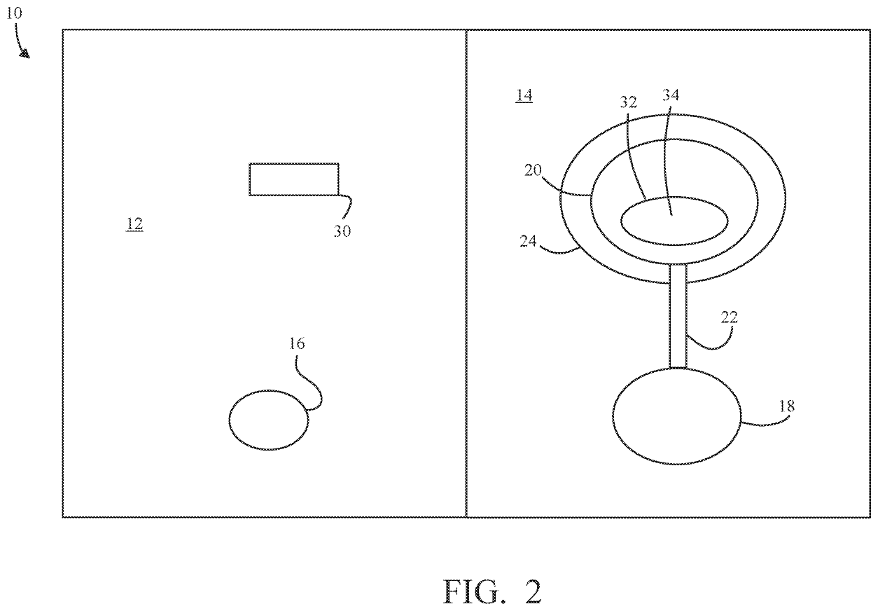

The passage 22 functions to facilitate directing the treated sample from the preamplification chamber to the first region 20. In some examples, the passage 22 may include one or more components configured to either treat the fluid sample or assist with amplification of the nucleotide sequence of the pathogen in the fluid sample before the fluid sample enters the first region 20. The passage 22 may have any shape or dimensions sufficient to allow the fluid sample to travel between the pre-amplification region 18 and the first region 20. The passage 22 may have a shape or dimension that is described with regard to channels or passages in relation to FIG. 2 or 4A-4B.

The heating element 24 functions to attain or maintain a temperature or range of temperatures sufficient to facilitate the amplification reaction and/or method. The heating element 24 may have any configuration sufficient to facilitate a desirable temperature for the amplification reaction. The heating element 24 may be directly connected with the first and/or second housings 12, 14 to reduce the number of loose components in the assembly 10, or in other examples, the heating element 24 may be a separate component that connects or is configured to simply contact a back side of the first and/or second housing 12, 14 so that different heating elements 24 could be implemented depending on the amplification method. The heating element 24 may comprise one or more components that work in combination to attain or maintain desirable temperatures. For example, the heating element 24 may include a battery and an electrical circuit that provides heat through running electricity through the electrical circuit. In another example, the heating element 24 may be configured as a chemical heater with one or more components that are configured react such that an exothermic or endothermic reaction progresses to adjust the temperature of the amplification reaction. Where the heating element 24 is configured as a chemical heating element, the chemical heating element may be positioned within a blister pack that is arranged such that upon puncturing or rupturing the blister pack, the chemical heating elements do not mix with amplification components of the first region 20. The heating element 34 may be configured such that the timing of the amplification reaction is adjusted so that a user can have a result in less time due to ideal heating conditions being used. Desirable temperatures, temperature ranges, and configurations for the heating element 24 are discussed herein.

The channel 26 functions to provide a medium for observing the results of the amplification reaction. In other examples, a portion of the channel 26 includes a lateral flow assay that can identify the presence of the pathogen. The channel 26 may be referred to, at least in part, as a second region where a user can identify whether a pathogen is present in the fluid sample. The channel 26 may be wholly visible through the window 30, or only a portion of the channel 26 may be visible through the window 30 such that the user can only see the portion of the channel 26 that is separated from the first region 20 by the plug 28. The channel 26 may have any shape and/or dimension sufficient to retain the plug 28 and to allow fluid flow when the plug 28 is dissolved, opened, or otherwise removed from the channel 26. For example, the channel 26 may have any configuration or dimension that is described in relation to the channels, passages, or pathways described in relation to FIGS. 4A-4B.

The window 30 functions to allow the user to visually determine whether a pathogen is present in the fluid sample. The window 30 may have any shape or configuration sufficient to observe the fluid sample before, during, or after the amplification method. The window 30 may include any material or component sufficient to allow the window 30 to remain transparent through the duration of and after the amplification method. In some examples, the window 30 extend across a portion or portions of the first housing 12 such that the first region 20, the preamplification region 18, and/or the channel 26. In some examples, the input 16 may be integrated with the window 30 so that the user can see the entire progression of the fluid sample through the assembly. In some examples, the window 30 may not be present, and the user identifies whether the pathogen is present by separating or rotating the first and second housings 12, 14.

FIG. 2 is a top view of an example of an assembly 10. The assembly 10 may be similar to the assembly 10 of FIG. 1. The assembly 10 includes the first and second housings 12, 14, the input 16, the passage 22, and the pre-amplification region 18, which may be similar to the housings 12, 14, the input 16, the passage 22, and the pre-amplification region 18 of FIG. 1. The input 16 connects with the pre-amplification region 18 and subsequently connects with the first region 20 via the passage 22. The first region 20 is surrounded, adjacent, and/or proximate by the heating element 24 for facilitating the temperature levels and ranges during the amplification reaction, and the heating element 24 may be similar to the heating element 24 of FIG. 1. The first region 22 surrounds the polymer 32 that is pH sensitive, and the polymer 32 or encapsulates the second region 34.

The second region 34 is configured to contain one or more dyes. The dye may be pH sensitive or inert, and the dye may be attached to a carrier. Any dye may be chosen that does not damage or otherwise interfere with the polymer 32 before the amplification reaction dissolves or becomes porous of the polymer 32. The first region 20 is configured to contain one or more components to assist with the amplification reaction. For example, the first region 20 may include one or more primers, buffers, chemical heaters, betaine, detergent, additional divalent cations, dNTPs, conjugated dNTPs (e.g., biotin-labeled dNTP), various enzymes such as reverse transcriptase, strand displacing enzymes common in isothermal amplification, lysing agents, or any combination thereof in solid state, liquid state, or both. As the fluid sample enters the first region 20, the amplification reaction begins to progress (assuming the pathogen is present). As the amplification reaction progress, the mixture of the amplification components react and acidity of the fluid sample begins to rise (i.e. pH drops) as a byproduct of the amplification reaction. As the pH of the mixture changes, the polymer 32 dissolves, becomes porous, or otherwise changes such that the dye within the second region 34 and the mixture of the amplification components and fluid sample can mix together to make a mixture that indicates the presence of the pathogen in the fluid sample. Where no pathogen is presence, the amplification components will not adjust the pH of the fluid sample, and therefore, the dye will remain stationary or confined within the second region 34 and will not mix with the components of the first region 20. Accordingly, the user can observe the results in the first region after a period of time through the window 30, which may be similar to the window 30 of FIG. 1.

The polymer 34 and/or plug 28 as described in relation to FIGS. 1 and 2 function to separate two regions. The polymers may be pH sensitive, temperature sensitive, or both such that upon activation of the amplification methods and increased acidity of the fluid sample the polymer becomes porous and/or dissolves. These amplification methods may utilize the intrinsic release by the amplification process of protons (i.e. acidification of the solution containing the amplification chemistry) to cause the polymers to be traversable barriers by liquids. Accordingly, the polymer indicates whether the amplification reaction has progressed and hence, when fluids traverse the polymer acting as a barrier, this is an indication of the pathogen. In some examples, the polymer is configured to contain a dye or gold nanoparticle. The gold nanoparticle may further participate in identifying the presence of a pathogen in a lateral flow assay as described herein. The dye and/or gold nanoparticle may be released from the polymer when the pH of the fluid sample adjusts such that the polymer dissolves or becomes more porous. The dye and/or gold nanoparticle may be contained in a single region and/or chamber that is surrounded by the polymer. The dye and/or gold nanoparticle may be integrated or encapsulated within the polymer. The dye and/or nanoparticle may be otherwise bound to the polymer and released from the polymer only after the amplification method adjusts the pH of the fluid sample. The polymer may have any structure that separates two regions and dissolves or becomes more porous at a certain pH. The polymer may be configured as a hydrogel, alcogel, microgel, or any combination thereof. Two different polymer compositions may be used in the same assembly depending on the desired configuration. For example, a first polymer that is sensitive to one range of pH may be used as a first barrier between regions, and a second polymer may be included downstream as a second barrier that is sensitive to a different pH than the first barrier. The polymer may be chosen based on whether the polymer melts, deforms, dissolves, or otherwise breakdown at temperature at or above a temperature of the amplification method. For example, the polymer may melt, deform, dissolve, or otherwise breakdown at a temperature of about 40 degrees Celsius or more, about 55 degrees Celsius or more, or about 70 degrees Celsius or more. The polymer may melt, deform, dissolve, or otherwise breakdown at a temperature of about 100 degrees Celsius or less, about 90 degrees Celsius or less, or about 80 degrees Celsius or less.

The polymer may be composed of any material sufficient to dissolve, become porous, or otherwise traversable as the pH of an adjacent fluid adjusts (i.e., becomes more acidic). For example, the polymer may include one or more of natural or semi-natural materials, synthetic acidic polymers, and synthetic basic polymers, or any combination thereof. the polymer may be basic or acid. The polymer may be a blend of different polymers. The polymer may be configured to respond to changes in pH at a specific range. For example, the polymer may be sensitive to a pH of 6 or more, 6.5 or more, or 7 or more. The polymer may be sensitive to a pH of 11 or less, 9.5 or less, or 7.5 or less. The polymer may include one or more of polycarboxylic acids, polyphosphoric acids, polyamino acids, polyboronic acids, poly vinylphenyl boronic acids, poly (3-acryl amido phenyl) acids, or any combination thereof. Polycarboxylic acids may include polyacrylic acid, poly methacrylic acid, polypropylacrylic acid, poly 4-vinylbenzoic acid, poly itaconic acid, or any combination thereof. Polyphosphoric acids may include one or more of poly(ethyl glycol acrylate phosphate), poly(vinyl phosphonic acid), poly(ethylene glycol methacrylale phosphate), poly(4-vinyl-benzyl-phosphonic acid), or any combination thereof. Polyamino acids may include one or more of poly (aspartic acid), poly (1-gluconic acid) poly (histine), or any combination thereof. Polyboronic acids may include poly (vinylphenyl boronic acid), poly(3-acryl amido phenyl acid), or any combination thereof. The polymer may include one or more of polymers containing tertiary amine groups, polymers containing morpholino or pyridine or piperazine groups, polymers containing pyridine or imidazole groups, and dendrimers.

Polymers may be either natural, chemically modified natural, synthetic, or mixtures of two. Examples of natural polymers may include but are not limited to chitosan, heparin, hyaluronic acid, and/or alginate. Chitosan may be a linear polysaccharide, natural water-soluble, and positively charged polymer with pKa of about 5.5 to about 7.5. Chitosan may be a copolymer of beta-(1-4)-linked D-glucosamino and N-acetyl-D-glucosamine. Because of the pKa of chitosan, chitosan offers a hydrogel structure with a neutral pH (i.e., 7.0) pH) and dissolves at acidic pH around 6.0 to 6.5. The physical properties of chitosan or other natural polymeric hydrogels can be modulated by chemical derivatization of the polymer. Examples of such derivatizations include but are not limited to acylation, aminization, and/or pegylation. In addition, the properties of the chitosan or natural polymeric hydrogels may be modified by percentage of the dissolved solid relative lo water in the hydrogel forming solution; by choice of molecular weight; and mixtures of molecular weights.

Polymers containing tertiary amine groups include but are not limited to: poly((2-dimethylamino)ethylmethacrylate), poly((2-diethylamino)ethylmethacrylate), poly((2-dipropylamino)ethylmethacrylate), poly((2-diisopropylamino)ethylmethacrylate), poly(N-(3-(dimethylamino)-propyl) methacrylamide), poly((2-dimethylamino)ethyl acrylate), poly(2-(tertbutylamino)ethylmethacrylate), poly(N,N-diakylvinylbenzylamine), poly((2-diethylamine)ethylacrylamide), or any combination thereof. Polymers containing morpholino or pyridine or piperazine groups may include poly((2-N-morpholino)ethylmethacrylate), poly(acrylomorpholine), poly((2-N-morpholino)ethylmethacrylamide), poly(Nethylprroidinemethacrlate), and poly(N-acylayl-N-alkenylpiperazine). Polymers containing pyridine or imidazole groups may include one or more of poly(4-vinylpyridine), poly(2-vinylpyridine), poly(Nvinylimidazole), poly(6-(1H-imidazol-1-yl) hexyl-methacrylate), or any combination thereof. Dendrimers may include one or more of poly(propylenimine) dendrimer, poly(ethylenimine) dendrimer, poly(amidoamine) dendrimer, or any combination thereof.

As used herein, blister packs may have any structure sufficient to contain one or more components or ingredients. The blister packs may include multiple chambers that include different ingredients that are designed to mix when the blister pack is ruptured. The blister pack may be configured to mix with components in another blister pack and/or location once the blister pack is ruptured. The blister packs may include solid components, liquid components, or both. The blister packs may include one or more, two or more, three or more, four or more, or a plurality of ingredients.

In other examples, the dye may be a pH sensitive dye that indicates a color change as the amplification method progresses. For example, no second region 34 and/or polymer 32 may be included, and all of the reactants and the dye may be positioned within the first region. Once the fluid sample is added to first region to form a mixture, the amplification method and/or reaction begins to progress, which would alter the pH of the mixture. Accordingly, the mixture would activate the dye that is pH sensitive (if the pathogen is present in the fluid sample), and the color change would be an indicator to the user of the assembly that the pathogen is present in the sample (i.e., a positive sample).

FIG. 3 is a side view of a housing 100. A first portion 102 is in contact with a filter 104 that separates the first portion 102 from a second portion 106. The first portion 102 is configured to receive saliva or another bodily fluid described herein from a patient, which then flows into the filter 104 and filters to the second portion 106. In the second portion 106, the saliva is mixed with a lysing agent to form a treated sample. Treated sample means saliva has been reacted or mixed with or filtered by one or more chemicals or filtration methods to ready the saliva for testing by one or more methods. At the second portion 106, a closable opening 108 allows fluids to flow through a bottom of the housing 100 when opened.

Initially, a patient deposits a saliva sample into the housing 100 for mixing with a lysing agent. As used herein, saliva or saliva sample can be stated as fluid sample, body sample, or patient sample. The housing 100 may be any container sufficient to housing 100 the lysing agent and the saliva so that a chemical reaction can ensue. For example, the housing 100 may be an Eppendorf tube, a conical vial, or any combination thereof. The housing 100 may have a length and/or a width sufficient to allow flow of the saliva and to house the filter 104. The length and/or width may be about 1 mm or more, about 5 mm or more, or about 1 cm or more. The length and/or width may be about 15 cm or less, about 10 cm or less, or about 5 cm or less. In some examples, the housing 100 may be integrated within an assembly at the input, such as the assembly 200 and input 204 of FIGS. 4A-4B. The housing 100 may have any shape sufficient to filter and process the saliva to form a treated sample. The housing 100 may include a cap that is configured to close the housing 100 and allow for agitation of the fluid sample. Upon emptying, the cap (not shown) may include a filter (not shown) that allows for filtering as the fluid sample is removed from the housing 100.

The filter 104 may function to extract oligonucleotides while minimizing byproducts. The filter 104 may include any compound sufficient to filter 104 oligonucleotides from the saliva sample. For example, the filter 104 may include aluminum oxide, or any combination thereof. The lysing agent may be integrated with the filter 104 in combination with other elements to assist with removing undesirable chemicals from the saliva. The filter 104 may have a nanopore size of about 10 nm or more, about 100 nm or more, or about 150 nm or more. The nanopore size may be about 500 nm or less, about 300 nm or less, or about 200 or less. The filter 104 may have a diameter the functions to match a diameter of the housing 100 so that the saliva is filtered as the saliva is deposited into a bottom of the housing 100. The housing 100 may include any number of filters 104 sufficient to extract oligonucleotides from the saliva to form a treated sample. The housing 100 may include one or more, two or more, three or more, or a plurality of filters. The housing 100 may have a closable opening at the bottom of the housing 100 that is connectable with the input 204 of the chip 202. When the closable opening is open, the treated sample (i.e., from the saliva) may deposit into the input of the chip for analysis. The bottom of the housing 100 may be configured to interface with the input so that loss of the treated sample is minimized. In some examples, no filter 104 may be included because no filtering is utilized with the treated sample.

The chemical reaction of the lysing agent and the saliva functions to extract nucleic acids from the saliva sample to form a treated sample that is used for input into the chip of the assembly. The lysing agent may function to lyse cells in the saliva and/or treated sample. For example, the lysing agent may include one or more of guanidine thiocyanate, lysozyme (e.g., Ready-Lyse Solution from Lucigen of Middleton, WI), lysostaphin, or any combination thereof. The lysing agent may be added to the saliva in any ratio sufficient to expose DNA material of a pathogen within a cell. The saliva and the lysing agent may have a ratio of about 4:1 or less to about 1:16 or more. The lysing agent may be contacted with the saliva before, after, or at the same time as when the saliva is contacted with the filter 104, described herein.

FIG. 4A is an exploded view of an assembly 200. FIG. 4B is a top view of a chip 202 of FIG. 4A. FIGS. 4A and 4B illustrate the chip 202 with a visible interior. To make the interior visible, the chip may be covered with a thin film that prevents interaction from the external environment including air, moisture, water, or other contamination. In some examples, the chip 202 is completely open, and reagents and/or samples flow through the chip 202 and are exposed to the external environment. In other examples, the chip 202 is made of one or more solid molds that allow for the internal structure of the chip 202 to be defined and do not allow a user or patient to see the flow pattern of the chip 202 from the outside of the chip 202. The chip 202 may have any length or width sufficient to provide a viable result of whether a pathogen is present in a saliva sample of a patient. For example, the chip may have a length and/or width of about 2 cm or more, about 4 cm or more or about 6 cm or more. The length and/or width may be about 20 cm or less, about 15 cm or less, or about 10 cm or less. The chip 202 may include any material sufficient to form a non-reactive and stiff structure to run the test for the pathogen. For example, the chip 202 may include one or more of polycarbonate, glass, polymethylmethacrylate, polydimethyl siloxane, metal, blends thereof, or any other rigid composition sufficient to house the fluids and facilitate the flow of the reactions described. The chip 202 may include any additive sufficient to reduce chemical interactions with the test and provide sufficient stiffness to allow fluids to flow unhindered through the chip 202. For example, the chip 202 may include one or more of UV stabilizers, fibers, fillers, a pigment, mold release agents, or any combination thereof.

The chip 202 defines an input 204 leading to a microfluidic channel 206. The input 204 and/or output 222 may be described as an opening or aperture. With the microfluidic channel 206, a treated sample (which is derived from saliva of a patient) is flowable from the input 204 to mix with amplification components positioned at a location of or adjacent to first reagent chambers 208a, 208b for amplifying the genetic material in the treated sample. The microfluidic channel 206 and/or chip may have a height that is uniform throughout length of the microfluidic channel 206 so that the treated sample is easily flowable to an end of the microfluidic channel 206. The microfluidic channel 205 may have any height and/or width sufficient to allow fluids to travel from the input 204 and through to the output 222. For example, the height and/or width may be about 0.1 mm or more, about 0.5 mm or more, or about 1.0 mm or more. The height and/or width may be about 2.0 mm or less, about 3 mm or less, or about 5 mm or less.

After the amplification reaction, which occurs within a first chamber 210, the treated sample is prepared to contact with assay material, which is stored in a second reagent chamber 212 and/or at the outlet 222 (i.e., lateral flow assay). A chamber as used herein may be a first, second, third, fourth, or fifth chamber. A chamber as used herein may be a main chamber, a secondary chamber, a reagent chamber, an amplification chamber, a component chamber, a lateral flow chamber, or any combination thereof. A channel may be any fluidic pathway of FIGS. 4A-4B. First, the treated sample may mix with a buffer simultaneously through a transition channel 214 when a separator 216 is removed from the slot 218, which opens the transition channel 214 for mixing the buffer and the treated sample within a second chamber 220. In other examples, the buffer may be positioned at a location of the second chamber 220, and the treated sample flows into the assay material in the second chamber 220. After mixing of the treated sample and the buffer, the combination of the treated sample and the buffer flow into an output 222, which includes an assay material integrated with the lateral flow assay that provides results of whether the treated sample (i.e., treated sample) contains the desired pathogen by showing test and control lines that are visible. The output as described herein may also be described as the lateral flow assay and include multiple subcomponents that visually indicate test and control lines.

The amplification components and buffers associated with the first and/or second chambers 210, 220 may be contained within blister packs positioned at the first and second reagent chambers 208a, 208b, 212. In other words, two blister packs containing the amplification components may be positioned at the first reagent chamber 208a, 208b and another blister pack may be positioned at the second reagent chamber 212. In the first and the second reagent chambers 208a, 208b, 212, a channel extends to the first chamber 210, the second chamber 222, or the transition channel 214. In other examples, the blister packs are positioned at a location of the first chamber 210 and/or the second chamber 220 so that the amplification components and/or buffer does not need to flow into any chamber to mix with treated sample.

To open the blister packs, an opener 224 is included that may be pivotably attached with the chip 202 at hinges 226a, 226b. The opener 224 includes spikes 228a, 228b that are configured to puncture or rupture the blister packs upon pivoting of the opener 224 on to a top surface of the chip 202. Specifically, the spike 228a configured to puncture, rupture, or pierce the blister pack positioned at the second reagent chamber 212, and the spike 228b is configured to puncture, rupture or pierce the blister pack positioned at the first reagent chambers 208a, 208b. In other examples, the opener 224 is not pivotably connected with the chip 202 and is a completely separate component (i.e., no connection points between the opener 224 and the chip 202). Additionally, the spikes 228a, 228b may have any configuration or number sufficient to puncture, rupture, or pierce blister packs positioned at locations of the chip 202. For example, the spikes 228a, 228b may align to blister packs positioned within the first and/or second chambers 210, 220.

The input 204 may function to receive the treated sample after saliva has been filtered and/or mixed with the lysing agent. The input 204 may have any shape or diameter sufficient to interface with the housing that stores the treated sample after mixing with the lysing agent. For example, the input 204 may have a shape of a circular, square, oblong, triangular, rectangular, oval, or any combination thereof. For example, the input 204 may have a diameter may be 2 cm or less, about 1 cm or less, or about 8 mm or less. The diameter may be about 1 mm or more, about 4 mm or more or about 6 mm or more. The input 204 may have a depth sufficient to form a connected with the microfluidic channel 206. For example, the depth may be about 0.5 mm or more, about 1 mm or more, or about 3 mm or more. The depth may be about 10 mm or less, about 7 mm or less, or about 5 mm or less. The input 204 may function as a pool or reservoir that temporarily holds a portion of the treated sample as the treated sample flows down the microfluidic channel 206 and gradually mixes with the amplification component.

The microfluidic channel 206 may function to facilitate movement of the treated sample from the input 204 to the output 222. The microfluidic channel 206 may have multiple portions along the chip 202. For example, the microfluidic channel 206 may have portions between the input 204 and the first chamber 210, between the first and second chambers 210, 220, between each of the first reagent chambers 208a, 208b and an entrance of the first chamber 210, between the second reagent chamber 212 and the second chamber 220, or between the second chamber 220 and the output 222. The microfluidic channel 206 may have any height or width sufficient to allow fluids to flow from the input 204 to the output 222. For example, the height and/or width may be about 0.1 mm or more, about 0.5 mm or more, or about 1 mm or more. The height and/or width may be about 5 mm or less, about 3 mm or less, or about 2 mm or less. The height and the width may be the same or different. The microfluidic channel 206 may have a shape along the cross-section of the microfluidic channel 206 that is circular, oval, oblong, square, rectangular, triangular, hexagonal, or any other shape sufficient to encourage fluid flow along the microfluidic channel 206. The transition channel 214 may have similar dimensions, shapes, and/or structures, as any portion of the microfluidic channel 206. For example, the dimensions, shapes, and/or structures of the transition channel 214 may be the same as the microfluidic channel 206. In other examples, the dimensions, shapes, and/or structures of the transition channel 214 may be different than the microfluidic channel 206.

The first and/or second chambers 210, 220 may function to facilitate either a reaction with or dilution of the treated sample so that the treated sample can be tested at the output 222 by a lateral flow assay. The first and/or second chambers 210, 220 may have any shape sufficient to facilitate dilution or reaction in the treated sample so that the treated sample can be tested. The first and/or second chambers 210, 220 may have a shape that is planar, rectangular, spare, circular, oval, oblong, hexagonal, pentagonal, triangular, or any other shape sufficient to facilitate dilution or reaction in the treated sample. The first and/or second chambers 210, 220 may include additional chambers and/or pockets that are configured to house blister packs and/or solid ingredients for mixing with the treated sample. For example, a solid amplification component may be positioned within a pocket of the first chamber, and the treated sample may flow into and mix or contact with the solid amplification component. In another example, a blister pack may be positioned at a location of or within the second chamber, and upon piercing, rupturing, or puncturing the blister pack and removing the separator, the treated sample may flow into and mix with the treated sample from the transition channel 214. In some examples, the first chamber 210 may include application components in solid and/or liquid form. The second chamber 220 and/or the outlet 222 may include assay material and/or buffer that is included in solid and/or liquid form.

The separator 216 may function to provide a mechanism for opening a fluid pathway between the first and second chamber 210, 220 or between the transition channel 214 and the second chamber 220. The separator 216 may be similar to the plug 28 and/or polymer 34 of FIG. 2. The separator 216 may have a length sufficient to extend across a first portion (at an intersection of first chamber 210a and the second chamber 220) and a second portion (at the intersection of the second chamber 220 and the second reagent chamber 212) of the transition channel 214 so that the separator 216 blocks the first and second portions until the separator 216 is removed. The separator 216 may have a dimension that matches the dimensions of the transition channel 214. The separator 216 may be sized to match the slot 218. The separator 216 and the slot 218 may be arranged or configured block the transition channel 214, and upon pulling the separator 216 a distance (e.g., 0.1 mm to 5 mm) out of the slot 218, apertures or channels (not shown) defined within the separator 216 may open the fluid pathway between the transition channel 214 and the second chamber 220.

The slot 218 may have a diameter and/or length sufficient to allow the separator 216 to slidably interface with the slot 218 to open the transition channel 214 to allow flow of the buffer in the second reagent chamber 212 and/or the treated sample in the first chamber 210. For example, the slot 218 may have a diameter of about 0.1 mm or more, about 1.0 or more, or about 3.0 mm or more. The slot 218 may have diameter of about 5.0 mm or less, about 20.0 mm or less, or about 10.0 cm or less. The slot 218 may have a length that matches the width of the chip or any other length sufficient to provide a closable mechanism for the transition channel 214 when the separator 216 is closed.

Once the treated sample enters the first chamber 210 or the first region 10, the treated sample mixes with the amplification component and an isothermal amplification reaction ensues that functions to amplify nucleic acids. The isothermal amplification reaction may include nucleic acid sequence-based amplification, loop-mediated isothermal amplification, helicase-dependent amplification, recombinase polymerase amplification, strand displacement amplification, rolling-cycle amplification, or any combination thereof. The isothermal amplification reaction may ensue for a period of time of 10 minutes or more, about 15 minutes or more, or about 20 minutes or more. The isothermal amplification reaction may ensue for about 3 hours or less, about 2 hours or less, or about I hours or less. The amount of time may be dependent on the external temperature of the environment. The isothermal amplification reaction may be conducted at room temperature (i.e., between about 20 degrees Celsius to about 30 degrees Celsius). Depending on the type of isothermal reaction used, the target compound may be RNA, RNA with reverse transcriptase, DNA, dsDNA, ssDNA, or any combination thereof. The techniques of isothermal amplification reaction may be applied to bacteria, viruses, biomarkers, plasmids, or any combination thereof.

In other examples, a heating element may be positioned at a location proximate or adjacent to the first chamber 210, such as how the heating element 34 is shown in FIGS. 1 and 2. With the heating element, the isothermal amplification reaction may be conducted at a temperature that is desirable, which may be a specific temperature or a range of temperatures. For example, the temperature may be about 20 degrees Celsius or more, about 30 degrees Celsius or more, or about 40 degrees Celsius or more. The temperature may be about 100 degrees Celsius or less, about 80 degrees Celsius or less, or about 60 degrees Celsius or less. The heating element may include a battery so that the assembly remains portable and does not require an external power source. The heating element may generate energy through a chemical reaction. This approach may be referred to as non-instrumented nucleic acid amplification (NINA). The NINA devices may be multi-ounce chambers. The chemical heaters may be reduced to simple blister-pack devices or the chemical heaters may be added to the amplification components in the first region and/or chamber. Examples of exothermic chemical reactions in the chemical heaters being employed may include CaO+H₂O, CaCl₂+H₂O, Fe+ (3) O₂ (in air), Na⁺ C₂H₃O₂, and MgFe+H₂O. In some examples, the exothermic chemistry of the heating element is contained within the first region and/or chamber and activated upon addition of the fluid sample. In some examples, the exothermic chemistry and the sample buffer or medium are chosen so as not to interfere with the isothermal amplification method. In other examples the exothermic chemistry is in an adjacent chamber with close thermal contact with the sample chamber. In such examples a method of initiating the reaction is included. In examples this method further comprises a blister pack, which ruptures a membrane enabling the powdered chemistry an aqueous solution to mix with the exothermic chemistry. In other examples the user adds an aqueous solution to the exothermic chemistry. The initiation of the chemical heating reaction may be an additional aliquot of the sample of bodily fluid. The heating element may be used to provide prolonged heat and maintain a temperature number or range of numbers over a period of time. In other examples, the heating element may expend substantially all of its stored energy at one time to quickly raise the temperature of the first chamber and/or first region.

The disclosed assemblies and methods may utilize one of two general amplification methods. The first may be an isothermal amplification method. The other may be a polymerase chain reaction. Both are described below with some more specific examples. In other examples, other amplification methods may be used in the context of the disclosed assemblies and methods as well.

Where Loop-mediated isothermal amplification (“LAMP”) is used, advantageous side effects of the reaction can be used to provide a positive or negative indication of the pathogen. Reverse Transcriptase Loop-mediated isothermal amplification (“RT-LAMP”) is modified to amplify RNA through the use of reverse transcriptase, which first converts viral RNA into cDNA. LAMP amplification is performed at constant temperature, usually between 60-65° C. LAMP uses four primers including two outer primers and two inner primers but may include one or more additional loop primers to accelerate the reaction. A strand displacing DNA polymerase, such as Bst DNA polymerase, Large Fragment or Bst 2.0 DNA polymerase, initiates DNA synthesis when combined with primers that recognize distinct target DNA sequences. A sustainable LAMP process includes the presence of six binding regions for four primers on the target DNA sequence, and results in high specificity. The forward and backward inner primers include a reverse complimentary sequence which results in formation of self-hybridizing loops. The forward and backward outer primers serve to displace the extending LAMP products, while the forward and backward loop primers facilitate exponential amplification of DNA Other combinations of primers may be used in the LAMP process to determine the presence of the desired pathogen as well.

LAMP reactions generally proceed in two steps. First, stem loop DNA structures are formed to serve as DNA templates for further amplification. Second, multimeric DNA molecules with inverted repeats are generated during amplification cycles forming large DNA concatemers. Amplified DNA products can be detected using visual methods, such as a change in color when using pH-sensitive dyes, or solution cloudiness (i.e., turbidity), but LAMP reactions can also be detected using various LFA (Lateral Flow Assay) approaches. DNA and RNA synthesis generally release hydrogen ions, H+ or H₃O+ as the LAMP reaction progresses. Thus, in the absence of suitable buffering, pH drops as the amplification reaction proceeds. This is advantageous because pH changes from LAMP can be used in combination with polymers or dyes to indicate the reaction is progressing due to the presence of a pathogen. For example, as described herein, a dye may be pH sensitive, and the progression of the LAMP reaction may activate the dye and indicate presence of a pathogen. In other examples, a polymer that is pH sensitive may be used and changes to the pH of the reaction may dissolve or make the polymer porous such that the fluid sample may flow to another region that provides indication of the pathogen, such as at a lateral flow assay or another chamber that contains a dye that can be mixed after the polymer opens or dissolves. In other examples, LAMP may produce large amounts of DNA amplicons when primers that recognize a target DNA sequence are included in the reaction mix. The reaction process may result in the production of pyrophosphate ions, which can bind to magnesium ions to form a white precipitate. This magnesium pyrophosphate precipitate can be quantified using turbidimetry to report the amount of nucleic acids present in a sample, which could be an indication that the pathogen is present in the fluid sample.

Where the recombinase polymerase amplification (“RPA”) is used, a special strand of the pathogen, along with other proteins (fully described in the relation to the amplification component), may be used to run several cycles of amplification. Specifically, for the actual biological process of RPA, recombinase binds with the primers, supported by the loading factors, to create a DNA-protein complex. These complexes then scan and “invade” the template DNA, hybridizing to their matches in the template and breaking the DNA at those points. Subsequently, ssDNA binding proteins help to stabilize this step of the reaction. Phosphocreatine, reacting with creatine kinases, produce ATP, which are hydrolyzed by the recombinase, leading to binding and activation of the strand displacing polymerase. The newly formed DNA then forms new complexes with recombinase that repeat the process, cycling until the system runs out of phosphocreatine (and therefore, is no longer able to produce ATP).

With this RPA technique, the amplification process may be performed at any temperature sufficient to amplify the DNA of the pathogen. For example, the temperature may be about 25 degrees Celsius or more, about 28 degrees Celsius or more, or about 32 degrees Celsius or more. The temperature may be about 44 degrees Celsius or less, about 40 degrees Celsius or less, or about 36 degrees Celsius or less. Where an external temperature of the assembly 100 or the temperature of the treated sample is not warm enough to facilitate the amplification reaction, a user, patient, and/or healthcare provider may hold the test with his or her hand to raise the temperature of the assembly 100 to a desirable number, as described above. The RPA test may be performed for any period of time sufficient to amplify the DNA of a pathogen. For example, the period of time may be about 15 minutes or more, about 20 minutes or more, or about 25 minutes or more. The period of time may be about 60 minutes or less, 45 minutes or less, or about 35 minutes or less. Where the RPA is used, the amplification components may include any component sufficient to amplify DNA of a pathogen.

The amplification component may function to amplify DNA of a pathogen for further testing of the presence of the pathogen in a treated sample. The amplification component may be stored on the assembly as a lyophilized pellet. The lyophilized pellet may be included directly with the assembly, or the lyophilized pellet may be added just before adding the fluid sample. In some examples, the amplification component is mixed with other components, such as chemical heaters or lysing agents, and the entire mixture is lyophilized. The lyophilized components can be resolubilized by the fluid sample and/or buffer. The amplification component may include one or more of rehydration and reaction buffer mixes (e.g, Tris(hydroxymethyl)aminomethane hydrochloride or phosphate buffered saline), primers, one or more reverse transcriptase enzymes, one or more DNA binding protein, one or more DNA polymerases, magnesium acetate or any combination thereof. The primers used in the amplification process may be any primer sufficient to identify genetic material of the targeted pathogen. The primer may be configured to bind with and/or identify a gene of a pathogen that is being targeted in the device. The primer may be configured to be complimentary to a gene of a pathogen that is being targeted. The primers may be selected based on the target pathogen and the desired amplification method. The assemblies and methods described herein may utilize any amount or combination of primers sufficient to identify whether the pathogen is present in a fluid sample. The primers may be configured to bind with a gene on a pathogen. The primers may be optimized for greater selectivity of a specific pathogen. In one example, the amplification component may include Twist Amp Basic DNA Amplification Kit from Twist DX of the United Kingdom. For example, the amplification component and the treated sample may be combined in any volumetric ratio sufficient to achieve isothermal amplification of the desired gene of the pathogen being targeted. The amplification component may be housed in any chamber proximate or adjacent to the first chamber 210. The amplification component may be in any form sufficient to perform the amplification process, such as in a form of a pellet, a solution, or in a combination of pellets and solution that mix upon entry of the treated sample. The amplification component may be housed in any vessel sufficient to hold a liquid amplification component, such as a blister pack. In other examples, the amplification component may be in a solid state form that is dissolved by the treated sample, as the treated sample flows in from the inlet. In some examples, some of the amplification components are in a liquid form housed in a vessel and some of the other amplification components are housed in a solid form within the first chamber 210, first region, or microfluidic channel.

In other examples, the isothermal amplification method may include one or more of the following techniques depending on the desirable configuration of the assembly: strand-displacement amplification (SDA); single primer isothermal amplification (SPIA); strand exchange amplification (SEA); cross-priming amplification (CPA); Helicase-Dependent Amplification (HDA); Rolling Circle Amplification (RCA); Multiple Displacement Amplification (MDA); Recombinase Polymerase Amplification (RPA); and Nucleic Acid Sequence-Based Amplification (NASBA).

In examples that do not use isothermal amplification, a polymerase chain reaction (“PCR”) may be used in place of the isothermal amplification process to amplify the desired nucleic acid sequence. Where PCR is a used, a heating element may be attached to the chip proximate or adjacent to the first chamber 210 for performing thermocycling. The thermocycling may be performed by a cycle of heating and cooling of the treated sample and a primer along a designated pathway (i.e., a serpentine pathway as illustrated in FIGS. 4A-4B). The PCR reaction may be performed for a period of time of about 1 hour or more, about 3 hours or more, or about 5 hours or more. The period of time may be about 10 hours or less, about 8 hours or less, or about 6 hours or less. Where a PCR is used, any component may be combined with a treated sample to amplify DNA for testing of the presence of the pathogen. For example, the PCR may include one or more amplification components described herein.

As a technique for confirming the presence of a pathogen in the fluid sample, a lateral flow assay (LFA) may be used downstream of the first chamber and/or region. The LFA may be conducted partially or wholly in the second region and/or chamber. The LFA may be configured to be performed downstream of an inlet and/or a polymer or plug. The LFA may have any configuration sufficient to indicate whether a pathogen is present in a fluid sample that has been amplified by one of the techniques described herein, such as isothermal amplification. The LFA may include a sample pad, a conjugate pad, a test line, a control line, and a wicking pad. In series, the sample pad is positioned first and will contact that fluid sample first. Then, the fluid sample travels down the conjugate pad and picks up gold nanoparticles, among other components described herein, and flows down to the test line and the control line, which indicate whether a pathogen is present. A wicking pad may be subsequently positioned at the end of the LFA to draw fluids from the sample pad and through the test line and the control line. The LFA may be paired with any amplification method described herein.

In other examples of LFA configurations, a modified LFA configuration may be paired with one or more arrangements described herein. For example, the fluid sample that is amplified in the first chamber and/or region may replace the sample pad. In another example, the pH-sensitive polymer may replace the conjugate pad by including gold nanoparticles that are released due to a temperature and/or pH change or the gold nanoparticles may be positioned adjacent to the pH-sensitive polymer and be carried down the modified LFA once the pH sensitive polymer is dissolved or made porous. In another example, the microfluidics can deliver the completed amplification reaction, which will interact with the gold-nanoparticles encapsulated in the pH-sensitive polymer, directly to a pad immediately adjacent to the test line.

In other configurations, diffusion of amplicons from the amplification reaction can interact with the gold nanoparticles in the LFA. While capillary action occurs when a liquid is pulled along a surface due to surface tension of the liquid, diffusion may occur when particles move from a high concentration region to an area of lower concentration. Diffusion may be facilitated in several ways including electrophoresis and pressure. Pressure may be applied to the reaction chamber and microfluidic channels during the action taken to initiate the reaction by breaking blister packs and mixing reactants. This may take the form of positive pressure from the sample side of the microfluidic channel, or negative pressure (suction) from the wicking pad side of the assembly. Diffusion may be facilitated through the use of a plunger that pushes (positive) or pulls (negative) generating pressure that will facilitate movement of the liquid in the assembly. Diffusion may also be implemented using a pump included in the assembly.

In a first example, the sample is configured with two labeled primers-one 5′-labeled with Biotin and one 5′-labeled with Fluorescein Isothiocyanate (FITC) serving as the forward and reverse primers. The primers may be used to amplify a specific section of the target DNA to create the labeled amplicon that now includes both labeled primers. The labeled amplicon will exist if the reaction is positive, and the target is present in the sample. This labeled amplicon may be included in the sample that is placed on the sample pad. Because of the presence of the wicking pad and the configuration of the device, the sample flows in one direction towards the wicking pad and across the test and control lines. The gold-nanoparticles conjugated to anti-FL antibodies may be positioned in the conjugate pad and as the sample passes from the sample pad through the conjugate pad to the test and control lines, the gold-conjugate binds to the FITC-labeled amplicons if the sample is positive, and to the free FITC labeled primer if there was no amplification. The test line may be made up of immobilized streptavidin (or avidin or neutravidin) that binds to biotin. If the sample is positive, and the dual-labeled amplicon exists, then the test line shows a positive response because the amplicon, which is also labeled with FITC will be bound with gold nanoparticles that creates the positive line. If the reaction is negative, free biotin labeled primers will bind to the test line but does not have the conjugated FL and therefore will not show a positive response. The control line may be made up of immobilized anti-rabbit-IgG (for example) that will bind to the free gold-nanoparticles conjugated to anti-FL antibodies. The control line will always appear because it will recognize and bind the gold-nanoparticles that are included in the Conjugate Pad.

In a second example, the sample may be configured with two labeled primers-one 5′-labeled with Biotin and one 5′-labeled with FITC serving as the forward and reverse primers. The primers may be used to amplify a specific section of the target DNA to create the labeled amplicon that now includes both labeled primers. The labeled amplicon will exist if the reaction is positive, and the target is present in the sample. This labeled amplicon may be included in the sample that is placed on the sample pad. Because of the presence of the wicking pad and the configuration of the device, the sample flows in one direction towards the wicking pad. The gold-nanoparticles conjugated to streptavidin (or avidin or neutravidin) are in the conjugate pad, and as the sample passes from the sample pad, through the conjugate pad, to the test and control lines, the gold-conjugate may bind to the biotin-labeled amplicons if the sample is positive and to the free biotin labeled primer if there was no amplification. The test line may be made up of immobilized anti-FL antibody that binds to the FITC. If the sample is positive, and the dual-labeled amplicon exists, then the test line shows a positive response because the amplicon, which is also labeled with biotin will be bound with gold nanoparticles that creates the positive line. If the reaction is negative, free FITC labeled primers will bind to the test line but does not have the conjugated biotin and therefore will not show a positive response. The control line may be made up of immobilized biotin that will bind to the free gold-nanoparticles conjugated to streptavidin. The control line will always appear because it will recognize and bind the gold-nanoparticles that are included in the conjugate pad.

In a third example, the sample may be configured as one labeled primer-5′-labeled with FITC, and biotin labeled dNTP (a deoxyribonucleotide triphosphate compound). Other triphosphate compounds may include deoxyadenosine triphosphate (dATP), deoxycytidine triphosphate (dCTP), deoxyguanosine triphosphate (dGTP), deoxythymidine triphosphate (dTTP), or deoxyuridine triphosphate (dUTP). The triphosphate compounds may be used in any LFA or amplification method described herein. The primer and biotin may be included in the reaction and are used to amplify a specific section of the target DNA to create the labeled amplicon that now includes both FITC and biotin. The labeled amplicon will only exist if the reaction is positive, and the target is present in the sample. In this situation, each amplicon may consist of multiple biotin labels that may result in signal amplification and greater sensitivity. This labeled amplicon may be included in the sample that is placed on the sample pad. Because of the presence of the wicking pad and the configuration of the device, the sample flows in one direction towards the wicking pad. The gold-nanoparticles conjugated to streptavidin (or avidin or neutravidin) may be included in the conjugate pad and as the sample passes from the sample pad, through the conjugate pad, to the test and control lines, the gold-conjugate binds to the biotin-labeled amplicons if the sample is positive and to the free biotin labeled primer if there was no amplification. The test line may be made up of immobilized anti-FL antibody that binds to FITC. If the sample is positive, and the dual-labeled amplicon exists, then the test line shows a positive response because the amplicon, which is also labeled with biotin will be bound with gold nanoparticles that creates the positive line. If the reaction is negative, free FITC labeled primers will bind to the test line but does not have the conjugated biotin and therefore will not show a positive response. The control line may be made up of immobilized biotin that will bind to the free gold-nanoparticles conjugated to streptavidin. The control line will always appear because it will recognize and bind the gold-nanoparticles that are included in the conjugate pad.

In some examples, a polymer may replace the conjugate pad. The polymer may include gold nanoparticle conjugates that are configured to participate in the LFA methods described herein. In some examples, the gold nanoparticles are released as the amplification method raises the pH of the fluid sample and either dissolves the polymer or makes the polymer more porous such that the fluid sample traverses the polymer and reaches the LFA After releasing the gold nanoparticle conjugates and traversing the fluid sample across the polymer, the LFA may progress via any method described herein.

The buffer associated with the first and/or second channel 210, 220 may function to improve the flow of fluids within the microfluidic channel 206, the first or second chambers 210, 220, the outlet 222, or any combination thereof. The buffer may be utilized in the first and/or second regions described in relation to FIGS. 1 and 2. The buffer may include any component sufficient to improve the flow of fluids within the microfluidic channel 206, the first or second chambers 210, 220, the outlet 222, or any combination thereof so that an adequate test result is received from the outlet 222. For example, the buffer may include one or more of phosphate buffered saline, tris(hydroxymethyl)aminomethane hydrochloride, a compound or solution containing saline, or a combination of both. The buffer may include a detergent or a surfactant to prevent non-specific sticking of components to one or more surfaces of the assembly. The components of the buffer may be dissolved in saline or water as a solvent. The buffer may be positioned at a location of the first and/or second reagent chambers 208a, 208b, 212, the transition channel 214, or the second chamber 220 so long as the treated sample is mixable with the treated sample before or after the amplification test. In other words, there may be a buffer associated with both the lateral flow assay and the amplification test, as required by the fluid dynamics of the microfluidic channel 206 or the first and/or second chambers 210, 220.

In the assemblies and methods described herein, a control reaction may be included to increase the confidence of the testing of a bodily fluid from a user. For example, using any amplification or detection or indication method described herein, a control reaction may be set up separately from the reaction that receives a fluid sample so that the reaction that receives the fluid sample is fluidly separate from the control reaction. The control reaction may function to indicate whether the amplification method and/or detection or identification method has properly progress. For example, a control sample of a known target DNA may be preloaded into the control reaction before being used by a user to test a bodily fluid. When the user inserts the bodily fluid into the assembly as described herein, the amplification and/or indication method may progress in the control reaction and the testing reaction. If the control reaction indicates the presence of the preloaded sample target DNA, then the user knows that the assembly is working properly, which provides a higher confidence in the result of the bodily fluid from the user.

The present assemblies and methods are directed towards testing for the presence of a pathogen in a portable assembly that does not require the use of instruments or long wait times to determine a result. The pathogen may be any virus or bacteria, such as those non-limiting examples described herein. Although some of the above examples are directed for testing for the presence of bacteria, ratios and/or reagents used could change depending on the pathogen being tested for. For example, the reagents, temperatures, timing, and/or ratios may be different if a virus is being tested for.

Non-limiting examples of pathogens that can be detected by the methods and assemblies disclosed herein include one or more various gram-positive bacteria such as Citrobacter freundii, Citrobacter diversus, Corynebacterium diptheriae, Diplococcus pneumoniae, Micrococcus sp. (I), Micrococcus sp. (II), Micrococcus sp. (III), Mycobacterium spp., Staphylococcus albus, Staphylococcus aureus, Staphylococcus citrens, Staphylococcus epidermidis, Streptococcus faecalis, Streptococcus pyogenes. Non-limiting examples include gram negative bacteria such as Acinetobacter calcoaceticus, Enterobacter aerogenes, Enterobacter aglomerans (I), Enterobacter aglomerans (II), Escherichia coli, Klebsiella pneumoniae, Nisseria gonorrhoeae, Proteus mirabilis, Proteus morganii, Proteus vulgaris, Providencia spp., Pseudomonas, Pseudomonas aeruginosa, Pseudomonas fragi, Salmonella choleraesuis, Salmonella enteritidis, Salmonella gallinarum, Salmonella gallinarum, Salmonella schottmuelleri, Salmonella typhimurium, Salmonella typhosa, Serratia marcescens, Shigella flexnerie Type II, Shigella sonnei, and/or Virbrio cholerae.

Non-limiting examples of viruses that can be detected by the techniques and assemblies as disclosed herein may include Coronaviridea including the subfamily Orthocoronavirinae (such as beta coronaviruses like SARS-CoV, SARS-CoV-2, MERS-CoV), as well as Adenovirus Type IV, Feline Pneumonitis, Herpes Simplex Type I & II, HIV-I (AIDS), Influenza A virus, Influenza B virus, Poliovirus, and/or Reovirus.

### Examples

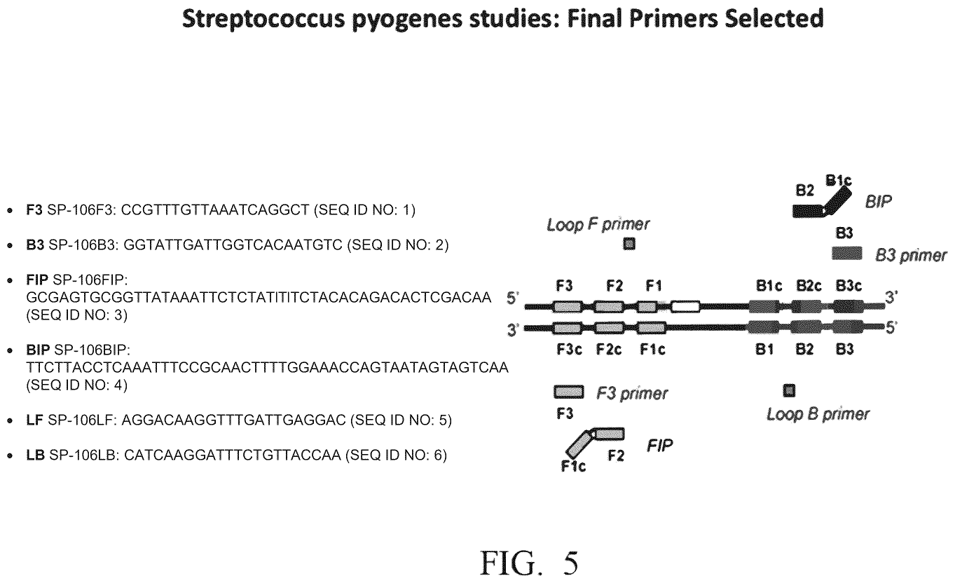

FIG. 5 shows a set of functional primers to carry out the LAMP reaction for the detection of Streptococcus pyogenes. LAMP uses a minimum of four primers, two outer primers and two inner primers, but may include one or two additional loop primers to accelerate the reaction. A strand displacing DNA polymerase, such as Bst DNA polymerase, Large Fragment or Bst 2.0 DNA polymerase, initiates DNA synthesis when combined with primers that recognize distinct target DNA sequences. A sustainable LAMP process utilizes the presence of six binding regions for four primers on the target DNA sequence, and results in high specificity. Typically, a six primer mix results in optimal LAMP reactions. The forward and backward inner primers include a reverse complimentary sequence which results in formation of self-hybridizing loops. The forward and backward outer primers serve to displace the extending LAMP products, while the forward and backward loop primers facilitate exponential amplification of DNA. LAMP reactions proceed in two distinct steps. First, stem loop DNA structures are formed to serve as DNA templates for further amplification. Next, multimeric DNA molecules with inverted repeats are generated during amplification cycles forming large DNA concatemers. Amplified DNA products can be detected using visual methods, such as a change in color when using pH-sensitive dyes, or turbidity, and LAMP reactions can also be detected using various LFA approaches.

To test of the presence of Streptococcus pyogenes, samples of saliva are combined with varying quantities of genomic DNA or bacterial pathogen in order to assess the amplification technique and minimum quantity of DNA/bacteria used in a sample for detection. This assessment is performed in separate phases; (1) genomic DNA in an inactivation buffer, (2) genomic DNA combined with saliva with/without inactivation buffer, and (3) live bacteria combined with saliva with inactivation buffer. Streptococcus pyogenes, the pathogen responsible for strep throat is used as the target genome, and Streptococcus agalactiae is used as the negative control.

### Genomic DNA and Buffer

Genomic DNA for Streptococcus pyogenes is acquired from American Type Culture Collection (ATCC) and combined with dilution buffer. Initial concentration is set at 1000 genome copies and compared to two negative controls in order to determine whether amplification using isothermal LAMP is possible. Color changes are used to assess amplification of the DNA in the sample, and a color change from purple to blue indicates a positive detection of target DNA. Amplification procedures are performed for durations ranging from 30 minutes to 40 minutes over a range of temperatures in order to determine desirable reaction conditions.

A limit of detection study is performed by conducting LAMP reactions at 61 degrees for forty minutes with varying amounts of genomic DNA ranging from 10 copies to 1000 copies. This was also performed at 61 degrees for 50 minutes, 60 minutes, 70 minutes, and 100 minutes. Based on colorimetric assessment, the limit of detection for a 40-minute reaction is found to be between 300 and 1000 copies. Color changes are observed at lower concentrations for extended reaction durations with non-specific signals detected by 100 minutes.

### Genomic DNA in Pooled Saliva

The next study consists of genomic DNA combined with human saliva. The temperature for all reactions is held at 61 C. Saliva is combined with genomic DNA of S. pyogenes at 1250 copies. Saliva is combined with genomic DNA in buffer at a ratio of 9:1 with and without inactivation buffer. DNA amplification is performed separately for 40, 45, and 50 minutes. Positive reactions are observed for all time points.

Limit of detection studies are then performed at 61 C for 40 minutes, using saliva and genomic DNA in inactivation buffer. 5 μL of pooled saliva from healthy individuals is added to 5 μL of inactivation buffer. Inactivation buffer is combined with genomic DNA at concentrations ranging from 0 to 5000 copies. Five replicates are performed for each DNA copy level. Positive reactions are observed in all samples at a concentration equal to or greater than 1250 copies. These results suggest that the limit of detection is between 625-1250 genomic copies for purified genomic DNA in pooled saliva.

Samples are assayed for optical density at 570 nm and 650 nm. OD values at each DNA concentration are averaged. Ratio of OD570/650 is used to determine quantitative positive and negative results, with a ratio greater than 1 indicating a positive result. These are compared to qualitatively observed color changes to validate positive and negative results. The optical measurements are consistent with the visible colorimetric results.

### Bacterial Detection in Saliva

Frozen bacteria are obtained for S. pyogenes and S. agalactiae with the latter serving as a negative control. Limit of detection studies are performed for colorimetric and optical analysis at 61 C for 35 or 40 minutes. 5 μL of healthy saliva is added to 5 μL of inactivation buffer containing varying numbers of bacteria ranging from O to 51,200 bacteria per sample. Positive results are observed in 100% of samples containing S. pyogenes bacteria, in concentrations as low as 200 bacterium per sample with the expected results observed for all negative controls.

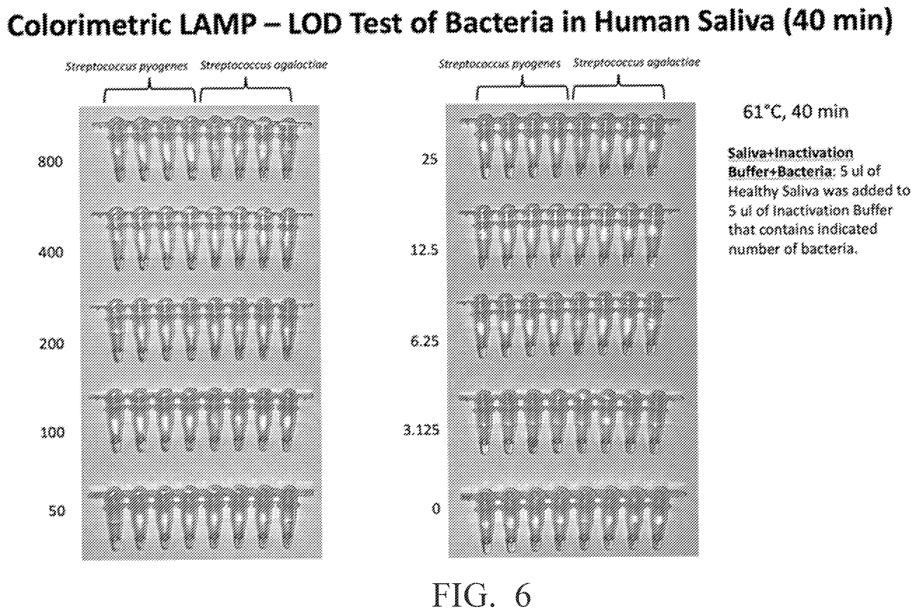

FIG. 6 shows the successful determination of Streptococcus pyogenes in human saliva using LAMP. To further characterize the limit of detection, this experiment is repeated with bacterial counts ranging from 800 down to 3.125 bacteria on average. Positive results were observed in all samples with a bacterial count greater than or equal to 6.25 when incubated at 40 minutes. These results indicate that the LOD for S. pyogenes using our current LAMP conditions is about 12-25 bacteria.

While the invention has been described in connection with certain embodiments, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

1. A method of detecting a pathogen in a fluid sample, comprising:
a. adding the fluid sample to a first region of an assembly;
b. amplifying a polynucleotide sequence of a pathogen in the first region using an amplification method; and
c. detecting a presence of the pathogen by:
i. identifying a release of a dye from a pH sensitive polymer after the amplification method adjusts a pH of the fluid sample; or
ii. identifying a release of a dye from the first region to a second region after the amplification method adjusts a pH of the fluid sample, wherein the first and second regions are separated by a pH sensitive polymer; or
iii. activating a pH sensitive dye in the first region to indicate a presence of the pathogen as the amplification method progresses; or
iv. indicating a presence of the pathogen on a lateral flow assay in a second region that is separated from the first region by a pH sensitive polymer so that the fluid sample traverses the pH sensitive polymer after the amplification method adjusts a pH of the fluid sample.

2. The method of claim 1, wherein amplifying the polynucleotide sequence of the pathogen in the first region using the amplification method comprises:
a. contacting the fluid sample with one or more amplification components and/or buffers; and/or
b. applying heat to the fluid sample to amplify the polynucleotide sequence.

3. The method of claim 1, wherein amplifying the polynucleotide sequence of the pathogen in the first region using the amplification method comprises:
a. contacting the polynucleotide sequence with a deoxyribonucleotide triphosphate compound so that acidity of the fluid sample is increased due to the amplification method.

4. The method of claim 1, wherein amplifying the polynucleotide sequence of the pathogen in the first region using the amplification method comprises:
b. contacting the polynucleotide sequence, one or more enzymes, one or more primers, and a deoxyribonucleotide triphosphate compound so that acidity of the fluid sample is increased due to the amplification method.

5. The method of claim 1, wherein the amplification method comprises polymerase chain reaction or isothermal amplification

6. The method of claim 1, wherein the isothermal amplification comprises at least one of loop-mediated isothermal amplification; strand-displacement amplification; single primer isothermal amplification; strand exchange amplification; cross-priming amplification; helicase-dependent amplification; rolling circle amplification; multiple displacement amplification; recombinase polymerase amplification; or nucleic acid sequence-based amplification.

7. The method of claim 1, where the amplification method comprises loop-mediated isothermal amplification.

8. The method of claim 1, wherein the pH sensitive polymer comprises natural or seminatural materials, synthetic acidic polymers, synthetic basic polymers, or any combination thereof.

9. The method of claim 1, wherein the dye comprises one or more of an organic dye and/or a synthetic dye, a nanoparticle, conjugated nanoparticle, and/or a quantum dot.

10. The method of claim 1, wherein the dye is attached to a carrier, and wherein the carrier comprises a protein, a polynucleic acid, a macromolecule, a virus particle, and/or a nanoparticle.

11. The method of any one of claim 1, where the pathogen is Streptococcus pyogenes.

12. An assembly for detecting a pathogen in a fluid sample, comprising:
a. a first region configured to perform an amplification method of a nucleotide of the pathogen, wherein the amplification method adjusts a pH of the fluid sample;
b. an indicator for determining presence of the pathogen, the indicator comprising:
i. a dye that is positioned within a pH sensitive polymer or is pH sensitive and within the first region, wherein the dye is configured to indicate presence of the pathogen as the amplification method adjusts the pH of the fluid sample; or
ii. a dye in a first region that is separated from the second region by a pH sensitive polymer so that the fluid sample is traversable across the pH sensitive polymer after the amplification method adjusts a pH of the fluid sample; or
iii. a lateral flow assay positioned within a second region, wherein the first and second regions are separated by a pH sensitive polymer so that the fluid sample is traverse-able across the pH sensitive polymer after the amplification method adjusts the pH of the fluid sample.

13. The assembly of claim 12, further comprising:
a. a cover that has at least one portion that is transparent and positioned over the first and/or second region so that indication of the pathogen is visible by viewing through the cover.

14. The assembly of claim 12, further comprising:
a. a heating element connected with the first region and configured to maintain a temperature or range of temperatures.

15. The assembly of claim 12, further comprising one or more buffers that do no inhibit adjustment of pH of the fluid sample, primers that interact with the nucleotide sequence of the pathogen, lysing agents that treat the fluid sample, or any combination thereof.
